(19) United States
(12) Patent Application Publication
(10) Pub. No.: US 2026/0077134 A1
(43) Pub. Date: Mar. 19, 2026

(54) INJECTION MONITORING MODULE

(71) Applicant: BIOCORP PRODUCTION S.A.S., Issoire (FR)

(72) Inventor: Alain MARCOZ, Issoire (FR)

(21) Appl. No.: 19/107,041

(22) PCT Filed: Sep. 29, 2022
(86) PCT No.: PCT/IB2022/000559

§ 371 Date: Feb. 27, 2025

(51) Int. Cl. A61M 5/315

(52) U.S. Cl. CPC A61M 5/31553; A61M 2205/0216; A61M 2205/3317; A61M 2205/581

(57) ABSTRACT

An injection monitoring module is adapted and configured to be removably mounted to an end of an injection pen having a body, a dose setting wheel connected thereto, and an injection activator. The dose setting wheel is rotatable about a central longitudinal axis of the pen injection system during dose setting and fixed against rotation during injection. The injection monitoring module comprises a hollow main body comprising a distal body portion, which extends around an outer surface of the injection pen body distally to the dose setting wheel, and the distal body portion comprises diameter altering means configured to dynamically alter an inner diameter of a central bore of the distal body portion from a first value to a second value different to the first value when mounting the injection monitoring module on the injection pen body.

The present invention relates generally to monitoring systems for injectable drug delivery devices, and in particular to injection monitoring for injection pen systems.

Injection monitoring is a well known field associated with injectable drug delivery devices, especially with regard to infusion systems, for example. Over time, such monitoring systems have been transferred more recently to injection pen systems for delivery of a drug, enabling users of such pen injection systems, and health care professionals involved in the treatment and follow-up of such patients, to monitor more closely their own injection regimes, and in many cases, the doses actually administered, in an attempt to lead to better healthcare outcomes. These developments have been accompanied by the increased associated use of software and portable communications devices such as tablets or smartphones, which have been programmed to receive information from, and interact with, the monitoring systems in order to provide information to the user or healthcare professional on-the-fly, or at regular intervals via appropriate communications units included in the monitoring systems.

In regard to pen injection systems in particular, for example, one of the challenges has been to provide easy to use, reliable and fairly failsafe systems that can be adapted to the various different variants of such commercially available pen injection systems, of which there are many. Previous attempts at providing such monitoring systems have usually involved adapting the body of the pen injection system by including electronic components therein along with one or more sensors. One of the major disadvantages of such systems however, is that they tend to make the end product, once all of the electronic components have been integrated, into fairly bulky and unwieldy objects, and thus more difficult to use from a user perspective. Additionally, such modified systems tend to be very specific to a given brand or a manufacturer, and thus of little or no use with other manufacturers. Furthermore, in order to overcome the issues with bulkiness and unwieldiness of the modified pen injection systems, there has been a tendency to attempt to reduce the overall volume of the injection pen bodies as much of possible through miniaturisation of the complex electronic components, which in turn has brought about its own problems, in particular with regard to electromagnetic interference between the various components due to the close proximities of the circuits providing the required or desired integrated functionality. Moving the sensors in such monitoring systems further away from the source of electromagnetic interference only further complicates matters, potentially leading to erroneous readings, or requiring further systems to compensate for the physical separation of the sensors from the other electronic components, such as a micro-controller designed to control and command the various components and manage their interactions.

The injection pen systems in question are well known per se and are commonly equipped with a proximally located dose setting wheel and injection activator, the dose setting wheel being rotatable about a central longitudinal axis of the pen injection system. The wheel is rotated by the user to select the dose of drug to be administered. The pen is generally configured, either mechanically or electro-mechanically to effect an injection upon activation of an injection activator. Such injection activators are quite commonly a simple press or push-button, in mechanical or electrical contact with the dispensing mechanism located within the pen injection system, the pressing of which causes the injection mechanism to fire and inject the drug contained within the pen injection system. In some pen injector systems, the dose setting wheel is configured to rotate not only during dose setting, but also during injection.

For example, PCT application published as WO2021/260404 discloses an injection monitoring module adapted and configured to be removably mounted to a proximal extremity of an injection pen system for delivery of a drug, the injection pen system having a pen body, a proximally located dose setting wheel connected to said body, and an injection activator, the dose setting wheel being rotatable about a central longitudinal axis of the pen injection system during dose setting and fixed against rotation during injection. The injection monitoring module, in particular, comprises:
a hollow main body adapted and configured to be coaxially mounted around the body of the pen injection system, the hollow main body comprising a central longitudinal bore having a proximal extremity and a distal extremity, and a central longitudinal axis;
a magnetic field production means, located on or within the hollow main body, at the proximal extremity of the central longitudinal bore;
an injection monitoring system comprising at least one or a plurality of magnetic sensors, the injection monitoring system being located at the proximal extremity of the bore of the hollow main body;
the hollow main body further comprising an inner sleeve located within the central longitudinal bore, and configured to frictionally engage with an outer surface of the dose setting wheel to co-rotate around the central longitudinal axis, without axial translation along said central longitudinal axis, with the dose setting wheel during during dose setting; wherein
the inner sleeve is connected to the injection monitoring system; and
the connection between the inner sleeve and the injection monitoring system is adapted and configured to co-rotate both the inner sleeve and injection monitoring system about the central longitudinal axis during dose setting, and to translate the injection monitoring system along the central longitudinal axis, but not rotate said injection monitoring system around said central longitudinal axis, during injection and/or ejection of a drug from the pen injection system.

As used herein, the terms “pen injection system” and “injection pen system” are used interchangeably to designate a generally handheld pen-shaped injection system, such systems being readily well known per se and commercially available for use in the treatment of many various medical indications. These systems are also often generally designed for self-injection of a drug by the user in need of treatment for the given medical indication, whereby the drug can be selected from a number of substances or combination of substances having a therapeutic or biological activity, to the extent that it has become a common occurrence for patients suffering from, or susceptible to, such medical indications to carry these devices around with them as and when might be required.

The injection pen system, to which the injection monitoring module is adapted and configured for removable attachment, is equipped with a proximally located dose setting wheel and an injection activator. The dose setting wheel rotates about a central longitudinal axis of the pen injection system to allow a user to set the dose of medicament for injection. The dose setting wheel is generally rotatable in both a clockwise, and a counter-clockwise direction, these directions corresponding generally to an increase in the selected dose, and a decrease in the selected dose, to be administered, respectively. The injection activator is often represented by a push-button, usually located proximally of the dose setting wheel, and in the majority of injection pens, at the proximal extremity of the injection pen system. After a dose has been set, and when a user of the injection system presses the injection activator in a distal direction, a piston is driven, generally mechanically or electromechanically, which is connected to a plunger in order to expel drug from a chamber within the injection pen body out through a needle that the user has inserted into an appropriate injection site, for example, the skin, fatty tissue, or muscle, depending on the type of drug to be administered. The dose setting wheel is often, but not necessarily, also coupled to the injection drive mechanism so that it also rotates as injection of the drug proceeds. The functioning of such injection systems is well known per se in the art. The monitoring module according as described in WO2021/260404 however, is mounted onto a pen injection system in which the dose setting wheel generally does not rotate during the ejection/injection phase of operation.

The injection monitoring module is adapted and configured to be removably attached to a proximal extremity of such an injection pen system. The expressions “removably attached”, “removably attachable”, “removably mounted” or “removably mountable” as might be used in the present specification are to be understood as referring to the possibility of attaching, or mounting, and subsequently removing, the injection monitoring module, for example, in the case of transferring the injection monitoring module to another pen injection system, or for example, if the monitoring module is damaged during use and requires replacement. Such attachment and subsequent removability can be achieved by providing coupling means on the monitoring module which engage in a releasable manner with the proximal extremity of the pen injection system, for example via frictional or elastic engagement, or via other releasable fastening means, such as clips, straps, screw threads and corresponding tightening rings, and the like, which engage with either the dose setting wheel, or the injection activator, or both.

The hollow main body of the injection monitoring module comprises a central longitudinal bore with a proximal extremity and a distal extremity, the bore being dimensioned to permit coaxial mounting of the hollow main body onto, and around the body of the pen injection system.

The hollow main body further comprises an inner sleeve located within the central longitudinal bore, and configured to frictionally engage with an outer surface of the dose setting wheel to co-rotate around the central longitudinal axis, without axial translation along said central longitudinal axis, but with the dose setting wheel during during dose setting, such that if the inner sleeve is rotated, then so does the dose setting wheel in the same direction, and to the same or identical degree of rotation. In this way, the inner sleeve can be said to co-rotate with the dose setting wheel.

The hollow main body is appropriately made of any suitable material, for example of a durable polymer or plastic material, such as high density or high impact polypropylene. Advantageously, the hollow body is made of transparent, translucent, or opaque material, in order to enable a user to apprehend and recognise any visual cues, such as light emitting diodes, that might also be provided or integrated into the injection monitoring module, where such cues can be optionally used to indicate various states of operation of the injection monitoring system. Similarly, the inner sleeve is also appropriately made of a suitable material, for example a durable polymer or high impact plastic material such as ABS.

The inner sleeve is moreover connected, or coupled, to the injection monitoring system. The connection, or coupling, between the inner sleeve and the injection monitoring system is adapted and configured to co-rotate both the inner sleeve and injection monitoring system about the central longitudinal axis during dose setting, and to translate the injection monitoring system along the central longitudinal axis, but not rotate said injection monitoring system around said central longitudinal axis, during injection and/or ejection of a drug from the pen injection system. To that end, the connection between the inner sleeve and the injection monitoring system is configured to selectively rotate about the central longitudinal axis, and then selectively translate along said longitudinal axis, the two movements being mutually exclusive of each other.

The hollow main body further comprises a distal body portion which extends around and frictionally engages with, an outer surface of the body of the injection pen system at a location distal to the dose setting wheel. In this way, the hollow main body is maintained in position on and around the body of the pen injection system distally of the dose setting wheel, which consequently is free to rotate within the bore of the hollow body. Such a frictionally elastic configuration can, for example, be provided via an appropriate elastomeric coating or deposit located on an inner circumferential surface of the hollow main body, for example in or more zones, or alternatively as a continuous, contiguous, or semi-continuous/contiguous coating deposited on said inner circumferential surface of the hollow main body. The objective of such a frictionally elastic coating or deposit is provide frictional grip between the distal body portion and the injection pen body in order to maintain correct positioning of the hollow distal body portion against the injection pen body. Appropriate types of elastomeric materials that can provide the correspondingly frictional engagement are known in the art per se.

The injection monitoring module also comprises an injection monitoring system comprising at least one or a plurality of magnetic sensors, the injection monitoring system being located at the proximal extremity of the bore of the hollow main body.

Notwithstanding the usefulness of the device described in WO2021/260404, the applicant has discovered that some pen injections systems on the market are provided with pen bodies which are shaped near the proximal end of the injection pen in such a manner that can lead to difficulties for a user in correctly mounting, or adjusting the mount position of, or respectively removing, an injection monitoring module as described in WO2021/260404, onto or respectively from the proximal end of the pen body, especially among populations of young, or physically frail, or otherwise physically impeded, users of those injection pen systems. For example, injection pen systems such as FlexTouch®, FlexTouch® Connect™, and Ozempic® are all provided with a pen body that has a portion of increased outer diameter at the proximal end of the pen body compared to the rest of the injection pen body, and compared to the respective outer diameters of the dose setting wheel and the activation button, which expanded diameter tapers off towards the distal extremity of the pen body to a smaller outer diameter.

The applicant proposes to address the disadvantages of the known injection monitoring modules, such as that described in the preceding paragraph by means of the present invention.

Accordingly, one aspect of the invention is an injection monitoring module adapted and configured to be removably mounted to a proximal extremity of an injection pen system for delivery of a drug, the injection pen system having a pen body, a proximally located dose setting wheel connected to said body, and an injection activator, the dose setting wheel being rotatable about a central longitudinal axis of the pen injection system during dose setting, wherein the injection monitoring module comprises:
a hollow main body adapted and configured to be coaxially mounted around the body of the pen injection system, the hollow main body comprising a central longitudinal bore having a proximal extremity and a distal extremity, and a central longitudinal axis;
a magnetic field production means, located on or within the hollow main body, at the proximal extremity of the central longitudinal bore;
an injection monitoring system comprising at least one or a plurality of magnetic sensors, the injection monitoring system being located at the proximal extremity of the bore of the hollow main body;
the hollow main body further comprising an inner sleeve located within the central longitudinal bore, wherein the inner sleeve is configured to frictionally engage with an outer surface of the dose setting wheel to co-rotate around the central longitudinal axis, without axial translation along said central longitudinal axis, with the dose setting wheel during during dose setting;
wherein the inner sleeve is connected to the injection monitoring system, and the connection between the inner sleeve and the injection monitoring system is adapted and configured to co-rotate both the inner sleeve and injection monitoring system about the central longitudinal axis during dose setting, and to translate the injection monitoring system along the central longitudinal axis, during injection and/or ejection of a drug from the pen injection system;
wherein the hollow main body further comprises a distal body portion which extends around an outer surface of the body of the injection pen system at a location distal to the dose setting wheel, said distal body portion comprising diameter altering means configured to dynamically alter an inner diameter of the central longitudinal bore of the distal body portion from a first value to a second value different to the first value.

According to another aspect, the diameter altering means of the distal body portion are configured to dynamically alter the inner diameter of the central longitudinal bore of the distal body portion from a first value to a second value smaller than the first value.

According to another aspect, the diameter altering means of the distal body portion are configured to dynamically alter the inner diameter of the central longitudinal bore of the distal body portion from a first value to a second value greater than the first value.

According to another aspect, the diameter altering means of the distal body portion are configured to dynamically alter the inner diameter of the central longitudinal bore of the distal body portion from a first diameter in which, when mounted on the injection pen device, the distal body portion is not in frictional contact with an outer surface of the injection pen device, to a second diameter, smaller than the first diameter, in which the distal body portion is in frictional contact with the outer surface of the injection pen device, said frictional contact preventing axial and radial movement of the distal body portion along and around the outer surface of the injection pen device.

From the above, it will be understood that the diameter altering means comprise one or more components which function, when operated, actuated, or activated, to dynamically alter the inner diameter of the central longitudinal bore of the distal body portion. The inner diameter of the central longitudinal bore, as referred to in the present specification, is considered to be the diameter which is defined, or definable, by the diameter formed by a radially inwardmost-facing surface portion of the distal body portion. The dynamic alteration of the inner diameter of the central longitudinal bore of the distal body portion is configured to produce, for example, as a function of an interaction between the components of the diameter altering means either a controlled or controllable reduction, or a controlled or controllable increase, of the inner diameter of the central longitudinal bore of the distal body portion. For example, the controlled reduction in diameter of the inner diameter of the central longitudinal bore of the distal body portion would be appropriate when fixing the injection monitoring module onto an outer surface of the injection pen system, and more particularly to the outer surface of an injection pen body. Similarly, and in the opposite sense, the controlled increase in diameter of the inner diameter of the central longitudinal bore of the distal body portion would be appropriate when releasing or unmounting the injection monitoring module from the outer surface of the injection pen system, and more particularly from the outer surface of the injection pen body, or when repositioning the injection monitoring module, should the need arise.

According to one aspect therefore, the diameter altering means comprises a first, rotatable outer component, and a second, non-rotatable inner component, wherein the diameter altering means is configured to dynamically alter the inner diameter of the central longitudinal bore of the distal body portion via a rotation of the first, rotatable outer component around and against the second, non-rotatable inner component.

The hollow main body comprising the distal body portion is advantageously comprised of a suitable material, such as a molded plastics material, for example, selected from the group consisting of ABS (acrylonitrile butadiene styrene polymer), PC (polycarbonate polymer), POM (polyoxymethylene momomer), and ABS-PC (acrylonitrile butadiene styrene polycarbonate co-polymer), whereby ABS-PC is advantageously preferred. Optionally, and advantageously, the hollow main body comprising the distal body portion can be at least partially made of a transparent or translucent material, such as one of those identified above, to facilitate visualization of an axial positioning of the injection monitoring module relative to the proximal end of the injection pen system when mounting the injection monitoring module onto the injection pen system.

According to another aspect, the first, rotatable outer component is a rigid outer ring. The rigid outer ring can also be made of one or more of the plastic materials identified above, but preferably and advantageously, will be made of a transparent plastic material such as polycarbonate polymer, and/or acrylonitrile butadiene styrene polycarbonate co-polymer.

According to another aspect, the second, non-rotatable inner component is suitably configured as a deformable inner ring. The non rotatable inner component, for exampled, when formed as a ring, is provided with a proximal end and a distal end, and a wall extending from the proximal end to the distal end, defining a bore having an inward facing surface defining an inner diameter, and an outward facing surface defining an outer diameter. The non rotatable inner component is physically connected to the hollow main body at a distal end of the hollow main body, for example, by welding or adhesion, for example, ultrasound welding, or alternatively through frictional surface engagement such as press-fit or snap-fit engagement, between a proximally-facing end surface of the non-rotatable inner component, and a distally-facing end surface of the hollow main body. In this way, the non-rotatable component is not permitted to rotate about the body of the pen injection system when the injection monitoring module is mounted on the pen injection system. As indicated above, the second, non-rotatable inner component is deformable. Generally speaking, and advantageously, the deformation of the second, non-rotatable inner component is configured to operate by reduction of the inner diameter of the second, non-rotatable inner component due to the application of a radially inwardly directed pressure on the outward facing surface of the second, non-rotatable inner component. This can be organized in a number of different ways, for example, the second, non-rotatable and deformable inner component, as exemplified by a ring, can be provided with at least one, or a plurality of, cut-away or removed portions of wall material, whereby the cut-aways or removed areas are distributed radially around the second, non-rotatable and deformable inner component, for example at equally and regularly spaced intervals around the diameter of the second, non-rotatable and deformable inner component, and preferably also extend along an axis perpendicular to the inner diameter of the second, non-rotatable inner component, for example, extending from a first proximal end of the deformable inner component at least part way along the wall in a distal direction, or alternatively terminating in a position adjacent to the distal end of the non-rotatable inner component. The removed, or cut-away portions of material allow the non-rotatable inner component to flex, or deform elastically, when radial pressure is exerted against the outward-facing wall surface of the non-rotatable inner component in a radially inward direction.

According to another aspect, the second, non-rotatable inner component, for example, formed as a deformable inner ring, comprises a radially inward-facing surface portion of elastomeric material. The elastomeric material can be distributed around and on, all, or just a part, of the radially inward-facing surface of the second, non-rotatable inner component. The elastomeric material is chosen to provide a frictional grip of the radially inward-facing surface of the second, non-rotatable inner component when the second, non-rotatable inner component comes into frictionally engaging contact with an outer surface of the injection pen system, for example, the pen body, and to thereby prevent the injection monitoring module from moving, or being moved, along the body of such an injection pen system, to an incorrect position along the pen body. Suitable elastomers for this task are thermoplastic elastomers, such as SEBS or polystyrene-poly(ethylenebutylene)-polystyrene block copolymer, and are known per se in the art.

According to another aspect, the radially inward-facing surface portion of elastomeric material has a first thickness at a first point along a radius of curvature of the inward-facing surface portion, and a second thickness different to the first thickness at a second point along the radius of curvature of the inward facing surface portion. The variation in thickness along a radius of curvature of the inward-facing surface portion is provided to enable the deformability configured into the second, non-rotatable inner component to be amplified for a relatively small effort applied to the outward-facing surface of the second, non-rotatable inner component. In this manner, relatively small applied forces to the outward-facing surface of the second, non-rotatable inner component will have a diameter altering effect that will vary not only according to the thickness of the elastomeric material along the radius of curvature, but also according to the diameter of an outward-facing surface of the injection pen body, thereby ensuring a perfect fit of the elastomeric material against the injection pen body.

According to another aspect, the first, rotatable outer component of the diameter altering means comprises at least one, or a plurality of, radially inwardly facing portion(s), and said one, or plurality of radially inwardly facing portion(s) are configured to bear down on the second, non-rotatable inner component and deform said inner component when said first, rotatable outer component is rotated around and against said second, non-rotatable inner component. It will be understood here that the first, rotatable outer component of the diameter altering means is therefore configured, via the presence of the at least one, or a plurality of, radially inwardly facing portion(s), to bring a force to bear against the second, deformable, non-rotatable inner component, and that this force causes deformation of the second, non-rotatable inner component as rotation of the first, rotatable outer component occurs.

According to another aspect, the at least one, or a plurality of, radially inwardly facing portion(s) of the first, rotatable outer component configured to bear down on the second, non-rotatable inner component are spaced equally one from each other around an inner circumference of the first, rotatable outer component.

According to another aspect, the at least one, or a plurality of, radially inwardly facing portion(s) of the first, rotatable outer component comprises three radially inwardly facing portions spaced equally apart.

According to another aspect, the radially outward facing surface of the second, non-rotatable inner component of the diameter altering means is configured to receive the at least one, or the plurality of, radially inwardly facing portion(s) of the first, rotatable outer component. It should be understood here that “configured to receive” means that the radially outward facing surface of the second, deformable, non-rotatable inner component of the diameter altering means is appropriately and suitably adapted, dimensioned, shaped, equipped, and/or provided with physically defined features that enable reception of the at least one, or the plurality of, radially inwardly facing portion(s) of the first, rotatable outer component by the second, inner component, in a manner such that the at least one, or the plurality of, radially inwardly facing portion(s) of the first, rotatable outer component physically engages with the radially outward facing surface of the second, deformable, non-rotatable inner component, and the latter is appropriately deformed through that physical engagement to alter the inner diameter of the second, inner component.

According to another aspect, the radially outward facing surface of the second, non-rotatable inner component of the diameter altering means comprises an annular groove, configured to receive, and engage frictionally, with the at least one, or the plurality of, radially inwardly facing portion(s) of the first, rotatable outer component.

According to another aspect, when rotated, the at least one, or the plurality of, radially inwardly facing portion(s) of the first, rotatable outer component engages with and bears against the annular groove of the radially outward facing surface of the second, non-rotatable inner component, and moves said at least one, or the plurality of, radially inwardly facing portion(s) of the first, rotatable outer component, about an axis of rotation of the first, rotatable outer component, from a first, radial position to a second, radial position which is different to the first radial position, whereby the inner diameter of the second, non-rotatable inner component is altered dynamically from a first inner diameter to a second inner diameter.

According to another aspect, the first, rotatable outer component comprises an alignment marker, which is positioned or located on an outward facing surface of the first, rotatable outer component, and which is configured to move, when the first, rotatable outer component is rotated about the axis of rotation, from a first radial position of non-longitudinal alignment with an alignment marker located on an outward facing surface of the hollow main body, to a second radial position of longitudinal alignment with said alignment marker located on the outward facing surface of the hollow main body. The alignment marker located on the first, rotatable outer component functions to provide a user with visual feedback and to indicate when the first, rotatable outer component is in a correct position for either dismounting the injection monitoring module from the injection pen body, or else to indicate that the injection monitoring module has been correctly positioned and fixed on the pen body ready for use to monitor dose setting and injection operations. The respective alignment markers can suitably be provided by, for example, appropriately shaped raised, or additional, portions of material located on an outward-facing surface of the respective rotatable outer component and hollow main body, or alternatively one or more coloured areas located on said outward-facing surfaces, or an appropriate combination of raised surfaces and coloured areas. For example, when the alignment marker of the first, rotatable outer component is in longitudinal alignment, i.e. with the alignment marker located on the outward facing surface of the hollow main body, along the length of the injection monitoring module, then the user knows that the injection monitoring has been correctly positioned and fixed, ready for use of the pen injection system and injection monitoring module. When the alignment marker of the first, rotatable outer component is not in longitudinal alignment with the alignment marker located on the outward facing surface of the hollow main body, then the user knows that the injection monitoring module is not yet ready for use, and optionally, may be in a position to be removed, or dismounted from the pen injection system.

According to another aspect, and alternatively, or in addition to the provision of alignment markers, or coloured areas, an indication to the user of a correctly reached position for securing, or being able to remove or dismount, the injection monitoring module to, or respectively from, the body of the injection pen, can be suitably provided via an alternative, or supplementary, positional feedback means distinct from the respective alignment markers or coloured areas. The first, outer, rotatable component and the second, inner, non-rotatable component can therefore be suitably shaped, configured and dimensioned to provide an alternative, or supplementary, positional feedback, for example, expressed as resistance to rotation, to the user, as the first, rotatable outer component reaches the permitted predefined limits of rotation about the central longitudinal axis. Each limit of rotation, and thus corresponding resistance to rotation, of the first, rotatable outer component, relative to the second, inner non-rotatable component, corresponds to a position in which the injection monitoring module is free to be removed or unmounted from the injection pen body, or oppositely, a position in which the injection monitoring module is securely fixed to the injection pen body and is incapable of any axial movement along, or radial or rotational movement around, the injection pen body. Alternatively, and or additionally, the alternative, or supplementary, positional feedback means can be provided by an audible signal, such as a click, for example, produced by a physical interaction of the first, outer rotatable component, against the second, inner non-rotatable component, such as a groove provided on one of the respective components, and a nub on the other respective component, of the distal portion, whereby the nub is configured to engage with, via elastic deformation, and be retained in, the groove at the point of, or substantially adjacent to the point of, greatest rotational resistance. Such an audible signal thus signifies to the user that a respective corresponding rotational stop position has been reached.

The invention will now be illustrated in more detail in reference to an example accompanied by the following drawings, in which:

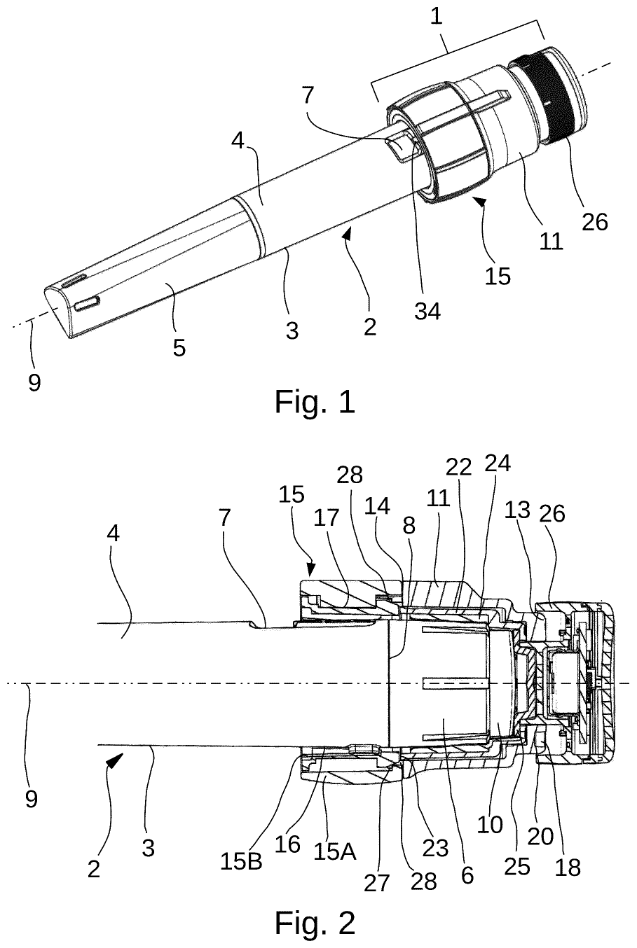

FIG. 1 is a schematic perspective representation of an injection monitoring module mounted on an injection pen system ready for use;

FIG. 2 is a schematic cross-sectional representation of the injection monitoring module when mounted on an injection pen system as illustrated in FIG. 1.

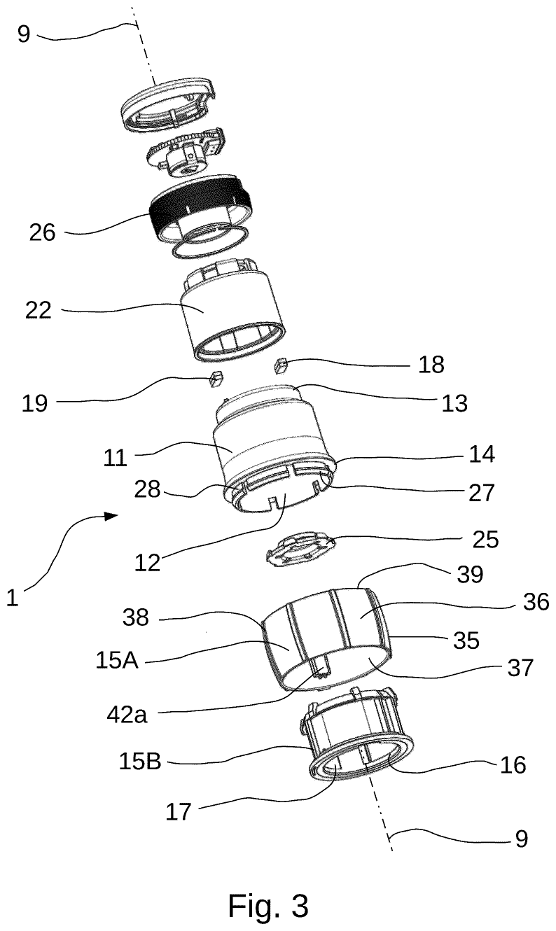

FIG. 3 is a schematic exploded perspective representation of the injection monitoring module of FIG. 1;

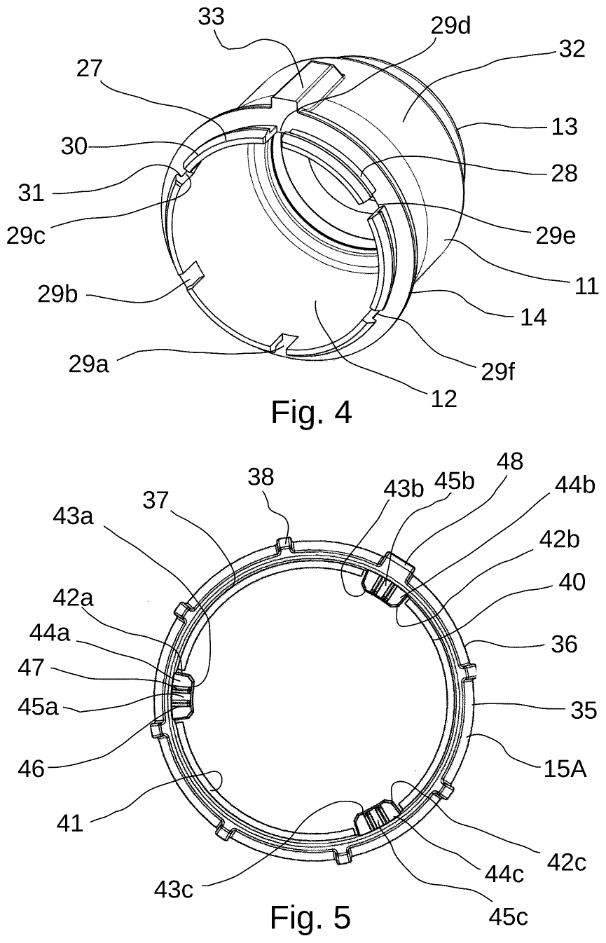

FIG. 4 is a schematic perspective representation of one part of the injection monitoring module of FIGS. 1 and 2;

FIG. 5 is a schematic end on representation of a part of the distal portion of the injection monitoring module of FIGS. 1 and 2;

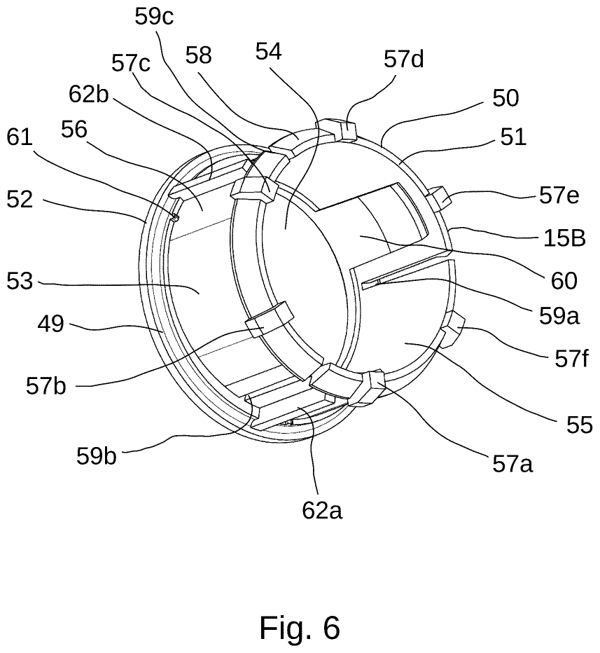

FIG. 6 is a schematic perspective representation of another part of the distal portion of the injection monitoring module of FIGS. 1 and 2;

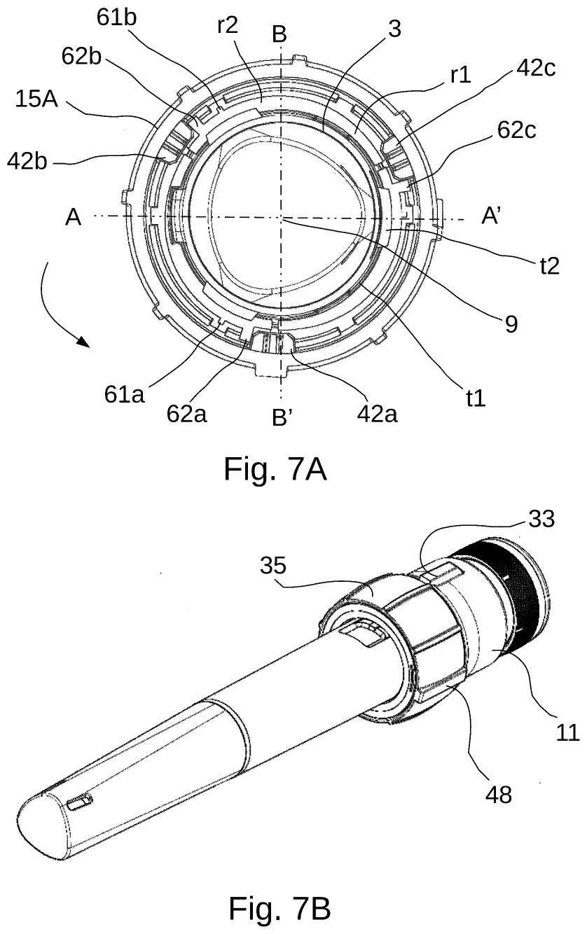

FIG. 7A is a schematic end on cut-away representation of the relative radial positions of the components of a distal portion of the injection monitoring module of FIGS. 1 and 2, in a first position;

FIG. 7B is a schematic perspective representation of an outward facing surface of the distal portion of the injection monitoring module according to FIG. 7A;

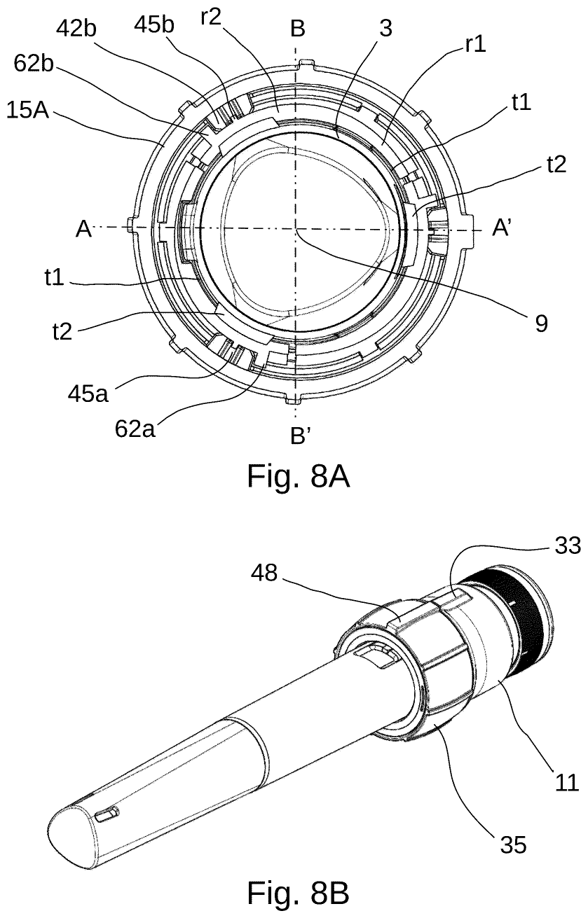

FIG. 8A is a schematic end on cut-away representation of the relative radial positions of the components of a distal portion of the injection monitoring module of FIGS. 1 and 2, in a second position;

FIG. 8B is a schematic perspective representation of an outward facing surface of the distal portion of the injection monitoring module according to FIG. 8A.

### DETAILED DESCRIPTION

Turning now to FIGS. 1, 2 and 3, schematic perspective, cross-sectional and exploded perspective representations of an injection monitoring module (1) according to the invention are shown. The injection monitoring module (1) is mounted on a handheld injection pen system (2), which comprises a pen injection system body (3) having an outer peripheral surface (4), a pen cap (5) covering the distal extremity of the pen injection system, a dose setting or dialling wheel (6), located at the proximal extremity of the pen injection system body (3), and a dialled dose visualisation window (7), located distally of the dose setting wheel (6), and displaying the dose which has been dialled by a user of the pen injection system. The injection monitoring module (1) according to the invention is located and adjacent to the proximal extremity (8) of the injection pen system (2), in particular at least partly around and in contact with the peripheral outer surface (4), surrounding and in contact with the pen body (3) and extending in a proximal direction beyond the proximal extremity (8) of the pen body (3) and in particular beyond the dose setting wheel (6). A central longitudinal axis (9) is also illustrated, which traverses the longitudinal axial centre of both the injection monitoring module (1) and the injection pen system body (3). The injection pen system (2) is provided with an activator button (10) proximally located from the dose setting or dialling wheel (6), as can be found in several commercially available injection pen systems. In the type of pen injection system displayed in FIGS. 1 and 2, the dose setting wheel is rotated about the central longitudinal axis (9) during dose setting, but is fixed against rotation during injection. A number of injection pens are currently commercialized which function in this manner, for example, Flex Touch® and FlexTouch Connect™, Ozempic®, and Norditropin® FlexPro®, all available from Novo Nordisk A/S.

The injection monitoring module (1) comprises a hollow main body (11) which is dimensioned and sized to be coaxially mounted around the body (3) of the pen injection system (2). The hollow main body (11) comprises a central longitudinal bore (12) having a proximal extremity (13) and a distal extremity (14), and a central longitudinal axis that coincides with the central longitudinal axis (9).

The hollow main body further comprises a distal body portion (15), which when mounted in a ready to use position, surrounds and frictionally engages with, the outer surface (4) of the body (3) of the injection pen system (2) at a location on the pen body (3) distal to the dose setting wheel (6). Frictional engagement of the hollow main body (11) with the outer surface (4) of the pen body (3) can be achieved by providing an elastomeric frictional material (16) on an inner peripheral surface (17) of the hollow main body. The hollow main body (11) extends in a proximal direction, above and beyond the limit of the activator button (10) of the pen injection system (2), such that the bore (12) houses both the dose setting wheel (6) and the activator button (10), and the dose setting wheel is free to rotate in the bore (12). The proximal extremity (13) of the bore corresponds so the proximal extremity of the hollow main body (11).

The hollow main body (11) further comprises a magnetic field production means (18, 19) located on or within the hollow main body (11), at the proximal extremity (13) of the central longitudinal bore (12). The magnetic field production means (18, 19) are suitably provided by a pair of single dipole magnets (18, 19), located diametrically opposite one to the other, each magnet respectively having a north (N) pole and a south(S) pole, with each pair of poles being preferably oriented in axial alignment from N-S along the central longitudinal axis, with the north pole being located proximally, and the south pole distally. The dipole magnets (18, 19) can be suitably formed into the shape of a rod, or alternatively as a disk or ring, or any other suitable shape. The magnets are located in a suitably dimensioned respective recess (20, 21) provided in the hollow main body (11), the recesses (20, 21) being located at the proximal extremity (13) of the body (11). Alternatively, the magnetic field production means can be a single dipole ring shaped magnet, which is seated on a peripheral proximal surface or within a corresponding annular recess of the hollow main body (11) at the proximal extremity (13) of said hollow main body. It will be understood from the above that the magnetic field production means do not rotate freely about the central longitudinal axis because the hollow main body (11), when the injection monitoring module is ready for use, is mounted on the pen body (3) around said central longitudinal axis (9) in a fixed positional relationship with regard to the pen body (3).

The hollow main body (11) further comprises an inner sleeve (22) located within the central longitudinal bore (12), and configured to frictionally engage with an outer surface of the dose setting wheel (6) to co-rotate around the central longitudinal axis (9), without axial translation along said central longitudinal axis, but with the dose setting wheel (6) during during dose setting.

The injection monitoring module is illustrated in a schematic exploded perspective representation in FIG. 3. In this view, the hollow main body (11), distal portion (15) and respective proximal (13) and distal (14) extremities are indicated. FIG. 3 also shows that the main hollow body (11) is shaped with a gradually widening inner diameter from the proximal extremity towards a point (23) adjacent, or in proximity, to the distal extremity (14) of the distal portion (15). This widening diameter corresponds to a widening of the bore (12) enabling the hollow main body to be inserted onto and around the proximal extremity of the pen and fit over the dose setting wheel (6) of the pen, whilst leaving sufficient room within the bore to receive the inner sleeve (22) so that the latter may engage with an outer surface of the dose setting wheel. The inner sleeve (22) is accordingly provided with a suitable contact, or engagement, surface (24) on an inward-facing side of the inner sleeve (22), and a corresponding contact, or engagement surface (25) is provided on an injection monitoring system housing (26), which engagement surface extends in a distal direction from the injection monitoring system housing into the bore (12), the engagement surface (24) and the engagement surface (25) cooperating to form an engagement surface which comes into frictional contact, and engages with respectively, an outer surface of the dose setting wheel (6), and the activation button (10).

The distal portion (15) is shown in FIG. 3 as a predominantly two-component system separate to the hollow main body (11), but as can seen from FIG. 2, it is pre-assembled together with the hollow body (11) when the distal portion (15) and hollow main body (11) are mounted on the body (3) of pen injection system (2). The distal portion (15) and hollow main body (11) can suitably be mated together during factory assembly of the injection monitoring module, for example, via spot welding of proximally facing surface of one of the components of the distal portion and distally facing surface of the hollow main body. Alternatively, and/or additionally, the hollow main body (11) at its distal end (14) can be provided with a radially outwardly extending distal annular skirt (27) and a distal annular wall (28) which extends from the hollow main body (11) and terminates in the radially outwardly extending distal annular skirt (27). The distal annular wall (28) has a reduced outer diameter compared to the outer diameter of the skirt (27). The distal body portion (15) is designed and configured to extend around an outer surface (4) of the injection pen body (3), at a location distal to the dose setting wheel. The distal body portion (15) comprises diameter altering means configured to dynamically alter an inner diameter of the central longitudinal bore of the distal body portion from a first value to a second value different to the first value. Accordingly, the diameter altering means provide for a controlled and dynamic reduction or increase of the inner diameter of the bore (12) in the distal body portion (15). In operation, the diameter altering means of the distal body portion are configured to dynamically alter the inner diameter of the central longitudinal bore of the distal body portion (15) from a first diameter in which, when mounted on the injection pen device, an inward-facing surface (17) of the distal body portion is not in frictional engaging contact with an outer surface (4) of the injection pen body (3), to a second diameter, smaller than the first diameter, in which the inward-facing surface (17) of the distal body portion is in frictional engaging contact with the outer surface (4) of the injection pen body (3), said frictional engaging contact preventing axial movement of the distal body portion (15) along the outer surface (4) of the injection pen body (3). As has been mentioned elsewhere in the present specification, the inner diameter of the central longitudinal bore is considered to be the diameter which is defined, or definable, by the diameter formed by the radially inwardmost-facing surface portion of the distal body portion (15).

Further details of features of the hollow main body (11) are illustrated in FIG. 4, of which the distal annular wall (28) and distal annular skirt (27) have already been identified and discussed. In addition, it can be seen from FIG. 4 that the hollow main body (11) is provided with multiple notches (29a, 29b, 29c, 29d, 29e, 29f) located in the distal annular skirt (27) and extending through the distal annular wall (28) from the distal end (14) to the distal extremity (30) of the annular skirt (27). The notches (29a-29f) are oriented along, and parallel to the central longitudinal axis (9), and advantageously spaced apart equally around the circumference formed by the distal annular skirt (27). The notches (29a-29f) are shaped, dimensioned and configured to receive, and engage with, correspondingly shaped, dimensioned and configured proximal elements of a part of the distal portion (15), as will be described hereafter with reference to FIG. 6. The distal annular skirt is further provided with a proximal-facing surface (31) which is configured, dimensioned, and shaped to provide a contact surface for a part of the distal portion (15) as described hereafter. The hollow main body (11) further comprises, located on a radially outward-facing surface (32) of the hollow main body (11), an alignment marker (33), extending radially outwardly from the radially outward-facing surface (32), and along and in parallel to central longitudinal axis (9). The length and height of the alignment marker can be adapted to suit the target user, and/or manufacturing constraints, as may be necessary. The alignment marker (33) serves as an indexing and positioning element for the hollow main body with regard to the dose visualisation window (7) of the injection pen body (3), when the hollow main body (11) is being mounted on the pen body (3), the dose visualisation window (7) generally being provided with a dose marker (34, FIG. 1).

In the embodiment presented in the figures, and visible in FIGS. 3, 5, 6, 7 and 8, the diameter altering means of the distal body portion (15) comprises a first, rotatable outer component (15A), and a second, non-rotatable inner component (15B), the inner diameter of the central longitudinal bore (12) in the distal body portion (15A/15B) being dynamically alterable via rotation of the first, rotatable outer component (15A) around and against the second, non-rotatable inner component (15B). The first, rotatable outer component (15A) is generally shaped, dimensioned, and configured as a rigid ring (35), having an outward-facing surface (36) and inward-facing surface (37). The outward-facing surface (36) can be provided with optional prehension means, such as at least one or more, or a plurality of, ridges (38), extending radially outwardly from the outward-facing surface (36) and aligned in parallel with the longitudinal axis (9) to allow a user to grip the rigid outer ring with the hand or digits, and rotate it about the longitudinal axis (9). At a proximal end (39) of the rigid outer ring (35), the ring (35) is provided with a radially-inwardly projecting shoulder (40) having a distal facing surface (41). When the distal portion (15) is assembled with the hollow main body (11), this distal facing surface (41) of the projecting shoulder (40) sits on, and slidingly engages with the proximal facing surface (31) of the distal annular skirt (27) in surface to surface engagement, enabling the rigid outer ring (35) to be rotated about the longitudinal axis (9) whilst being supported by the proximal facing surface (31) of the distal annular skirt (27). The rigid outer ring (35) is also provided with at least one (cf. FIG. 1) or, as illustrated in FIG. 5, a plurality of, radially inwardly facing and extending portions (42a, 42b, 42c), each portion (42a, 42b, 42c) defining a respective inward facing surface (43a, 43b, 43c) which faces into the bore (12). As exemplified in FIG. 5, there are three, radially inwardly facing portions (42a, 42b, 42c) and corresponding inward-facing surfaces (43a, 43b, 43c), but fewer than three or more than three could be provided, as deemed necessary. Each of the radially inward-facing and radially inward extending portions (42a, 42b, 42c) and corresponding inward-facing surfaces (43a, 43b, 43c) are shaped, dimensioned and configured to bear down on the second, non-rotatable inner component (15B) and deform the latter when the rigid outer ring (35) is rotated around and against the second, non-rotatable inner component (15B). It will be understood therefore that the inward-facing and radially inward extending facing portions (42a, 42b, 42c) and corresponding inward-facing surfaces (43a, 43b, 43c) bring a force to bear against the second, deformable, non-rotatable inner component (15B), and that this force causes deformation of the second, non-rotatable inner component (15B) as rotation of the rigid outer ring (35) occurs. Advantageously, each inward-facing and radially inward extending portion (42a, 42b, 42c) are spaced equally apart around the circumference of the inward facing surface (37) of the rigid outer ring. As can also be seen from FIG. 5, each inward-facing and radially inward extending portion (42a, 42b, 42c) has a distal-facing surface (44a, 44b, 44c), in which a seating recess (45a, 45b, 45c) is formed or provided, for example, as defined by a pair of parallel extending ridges (46, 47) of material extending from the distal-facing surface (44a, 44b, 44c) in a distal direction. The rigid outer ring (35) is further provided with an alignment marker (48) on the outward-facing surface (36) of the ring (35), which serves to visualize alignment or non-alignment of the rigid outer ring (35) with the alignment marker (33) of the hollow main body (11), Non-alignment between the two markers (33, 48), in parallel to the longitudinal axis (9) of injection monitoring module, signifies that the injection monitoring module (1) is not securely mounted to the pen body (3), and that the possibility exists for the injection monitoring module (1) to be removed from the injection pen (2), whereas alignment of the two markers (33, 48), in parallel to the longitudinal axis (9) of the injection monitoring module (1), signifies that the injection monitoring module (1) is securely mounted to the injection pen body (3) and is ready for use in monitoring an injection.

As illustrated in FIG. 6, the second, non-rotatable inner component (15B) is configured as a deformable inner ring (49). The deformable inner ring (49) has a proximal end (50) with a proximal-facing surface (51), and a distal end (52), with a wall (53) extending from the proximal end (50) to the distal end (52), defining a bore (54) having an inward-facing surface (55) defining an inner diameter, and an outward facing surface (56) defining an outer diameter. The deformable inner ring (49) is physically connected to the hollow main body (11) at the distal end (14) of the hollow main body (11), via a series of one or more noses (57a, 57b, 57c, 57d, 57e, 57f), which extend and project in a proximal direction beyond the proximal end (50) and proximal facing surface (51). The noses (57a-57f) engage via elastic frictional engagement respectively with, and/or are welded or adhered, for example, by ultrasound welding, to the notches (29a-29f). The proximal end (50) and noses (57a-57f) can further be shaped with a frustoconical surface (58) with a widening taper from the proximal end (50) in the distal direction, to ease insertion into, reception by, and engagement with, the notches (29a-29f). In this way, the non-rotatable deformable inner ring is prevented from rotating about the injection pen body (3) when the injection monitoring module (1) is mounted on the pen body (3). As indicated above, the inner ring (49) is deformable. The deformation of the inner ring (49) is assisted through the provision of at least one, or as illustrated in FIG. 6, a plurality of, cut-away or removed portions (59a, 59b, 59c) of wall (53) material, whereby the cut-aways or removed areas are distributed radially around the deformable inner ring (49), for example at equally and regularly spaced intervals around the circumference of the inner ring (49). The cut-away portions (59a, 59b, 59c) preferably also extend along an axis perpendicular to the inner diameter of the inner ring (49), for example, extending from the proximal end (50) of the deformable inner component at least part way along the wall (53) in a distal direction, or alternatively terminating in a position adjacent to the distal end (52) of the non-rotatable inner ring (49). The removed, or cut-away portions (59a, 59b, 59c) of wall (53) material allow the non-rotatable inner ring to flex, or deform elastically, when radial pressure is exerted against the outward-facing wall surface (56) of the non-rotatable inner ring in a radially inward direction, and to flex radially outwardly again to a default position when such radial pressure is removed or lessened.

As mentioned above, the deformable inner ring (49), comprises a radially inward-facing surface (55), which is provided with, and at least partly covering said inward-facing surface (55), a portion of elastomeric material. The elastomeric material can be distributed around and on, all, or just a part, of the radially inward-facing surface (55) of the second, non-rotatable inner ring (49). The elastomeric material is chosen to provide a frictional grip for the radially inward-facing surface (55) when the non-rotatable inner ring (49) comes into frictionally engaging contact with the outer surface (4) of the injection pen body (3), thereby preventing the injection monitoring module (1) from moving, or being moved, along the body (3) to an incorrect position. Suitable elastomers for this task are thermoplastic elastomers, such as SEBS or polystyrene-poly(ethylenebutylene)-polystyrene block copolymer, and are known per se in the art.

The elastomeric material has a first thickness (t1) at a first point (r1) along a radius of curvature of the inward-facing surface (55), and a second thickness (t2) different to the first thickness (t1) at a second point (r2) along the radius of curvature of the inward facing surface (55). The variation in thickness along a radius of curvature of the inward-facing surface (55) is provided to enable the deformability, which is configured into the non-rotatable inner ring (49) to be amplified for a relatively small effort applied to the outward-facing surface (56) of the wall (53) of the non-rotatable inner ring. In this manner, relatively small applied forces to the outward-facing surface (56) of the wall (53) of the non-rotatable inner ring will have a diameter altering effect that will vary not only according to the thickness of the elastomeric material along the radius of curvature, but also according to the diameter of the outward-facing surface (4) of the injection pen body (3), thereby ensuring a snug fit of the elastomeric material against the injection pen body (3).

The deformable, and non-rotatable, inner ring (49) is further provided with at least one seating nub (61a, 61b, 61c), located for example near the distal end (52) on the outward facing surface (56) of the wall (53) of the inner ring (49). The seating nub is shaped, dimensioned and configured to fit in the recesses (45a, 45b, 45c) provided on the distal-facing surfaces (44a, 44b, 44c) of the inward-facing and radially inward extending portions (42a, 42b, 42c), and serves to temporarily hold the inward-facing and radially inward extending portions (42a, 42b, 42c) in a given radial position about the central longitudinal axis (9) when no rotational force is being applied to the rigid outer ring (35).

The inner deformable, and non-rotatable ring (49) is also provided with rotational stops (62a, 62b, 62c). The rotational stops extend from adjacent the proximal end (50), for example, distally to the frustoconical surface (58) of the proximal end, in the direction of the distal end (52). The rotational stops (62a, 62b, 62c) also extend radially outwards, thereby defining a raised transverse bar which extends over the outward facing surface (56). Each rotational stop (62a, 62b, 62c) defines, with an adjacent rotational stop (62a, 62b, 62c), an arc of permitted movement of the inward-facing and radially inward extending portions (42a, 42b, 42c) of the outer ring (35) about the axis (9). The stops are positioned around the circumference of the outward facing surface (56) of the wall (53) of the inner ring in such a manner that the corresponding position of the inward-facing and radially inward extending portions (42a, 42b, 42c) of the outer ring (35) exerts either a minimum, or respectively, a maximum of radially directed force onto the outward-facing surface (56) of inner, deformable ring, causing said ring to be either in minimum radial compression, or maximum radial compression, respectively. In other words, the rotational stops (62a, 62b, 62c) and corresponding proximal (50) and distal (52) ends of the inner deformable, and non-rotatable ring (49) define an annular groove, separated into a series of arcuate portions of permitted rotational movement of the inward-facing and radially inward extending portions (42a, 42b, 42c) of the outer ring (35).

The functioning of the distal portion will now be described with particular reference to FIGS. 7A, 7B, 8A and 8B. FIG. 7A shows an end on cut-away view of the injection monitoring module (1) in a first, unfixed position, around the pen body (3), and the relative radial positions of the various elements of the rotatable, rigid outer ring (35) and the non-rotatable, deformable, inner ring. FIG. 7B shows a perspective view of the injection monitoring module (1) mounted, but not fixed, on the injection pen body (3). The lack of fixed mounting is indicated by the absence of alignment between the alignment marker (33) of the hollow main body (11) and the alignment marker (48) located on the outer surface (36) of the outer ring (35). In these relative positions, one can see from FIG. 7A that the inward-facing and radially inward extending portions (42a, 42b, 42c) of the outer ring (35) do not radially engage, bear down, or press against the inner ring (49), or if they do so engage, then it is with minimum inward radial force. For example, it can be seen that the inward-facing and radially inward extending portion (42c) is located in rotational abutment against the rotational stop (62c), but that in this position, it does not exert any radially inward force on the outward-facing surface (56) of the inner ring (49), nor on the reduced thickness portion t1 at radial point r1. The arrow to the left of FIG. 7A indicates the direction of rotational force or effort to be imparted by a user on the outer ring (35) when they wish to fix the distal portion (15) and injection module (1) as a whole to the injection pen body (3). In this figure the rotation is counter-clockwise, but the distal portion could be configured to function in the opposite direction, i.e. by imparting a clockwise rotational effort or force on the outer ring (35). As the user rotates the outer ring in a counter-clockwise direction, the inward-facing and radially inward extending portion (42a, 42b, 42c) rotate in a counter-clockwise direction. In so doing, they follow the arc of the groove defined between the rotational stops (62a, 62b, 62c) and the outward-facing surface (56) of wall (53), and come to bear down on, and against, the outward-facing surface (56) of the wall (53), moving into contact with, and bearing down on the portion of increased thickness t2 at radial point r2. The wall (53) deforms under the radial inward pressure at the same time, due to the plurality of cut-away or removed portions (59a, 59b, 59c) of wall (53), thereby decreasing in inner diameter. The decrease in inner diameter of the bore causes the elastomeric portion located on the inward facing surface (55) of inner ring to come into elastic and frictionally engaging contact with the outward facing surface (4) of the pen body, thereby securing the distal portion to said pen body (3).

FIGS. 8A and 8B illustrate the secured, or fixed, mounting of the injection module (1) with its distal portion in the correctly aligned position on the pen body (3), and the relative radial positions of the elements of the distal portion (15). In FIG. 8A, for example, one can see that the inward-facing and radially inward extending portions (42a, 42b, 42c) of the outer ring (35) all bear down on the outer surface (56) of the inner ring (49) at a position (r2) around the axis (9) at which the thickness of the elastomer is at a maximum (t2). Additionally, the inward-facing and radially inward extending portions (42a, 42b, 42c) of the outer ring are in rotational abutment against the rotational stops (62a, 62b, 62c) and are additionally simultaneously seated on the seating nubs (61a, 61b, 61c) of the inner ring via engagement of the nubs in the recesses (45a, 45b, 45c) provided on the inward-facing and radially inward extending portions (42a, 42b, 42c) of the outer ring. FIG. 8B illustrates that in these relative positions, the user is informed that the injection monitoring module is securely mounted on the pen body due to the visible alignment between the alignment marker (33) of the hollow main body (11) and the alignment marker of the outer ring (35) of the distal portion (15). Additionally, as the outer ring is rotated into the rotational stop position, the movement of the inward-facing and radially inward extending portions (42a, 42b, 42c) and recesses (45a, 45b, 45c) of the outer ring against the seating nubs (61a, 61b, 61c) of the inner ring provides a physical, or haptic, feedback to the user. This physical, or haptic feedback is experienced by the user as an increased resistance to rotation, as the ridges (46, 47) defining the recesses (45a, 45b, 45c) are pushed against, and over the seating nubs (61a, 61b, 61c), with the ridges (46, 47) deforming elastically to allow the seating nubs to engage elastically in the recesses (45a, 45b, 45c), and thereby generating an audible signal, such as a clicking sound. The clicking sound is thus a form of feedback that indicates to the user that the distal portion of the injection monitoring module is either securely fixed to the injection pen body, or when the nubs (61a, 61b, 61c) engage in the recesses (45a, 45b, 45c), in the opposite rotational stop position, that the distal portion of the injection monitoring module is able to be dismounted from the injection pen body. Thus, in order to unmount the injection monitoring module (1) from the pen body (3), the outer ring is rotated in the opposite direction, i.e. in this illustrated example, in a clockwise direction to release or remove the inward radial force of the outer ring from bearing down on the inner ring, allowing the inner ring to return to its default, nominal dimensions.

1. Injection monitoring module adapted and configured to be removably mounted to a proximal extremity of an injection pen system for delivery of a drug, the injection pen system having a pen body, a proximally located dose setting wheel connected to said body, and an injection activator, the dose setting wheel being rotatable about a central longitudinal axis of the pen injection system during dose setting and fixed against rotation during injection, wherein the injection monitoring module comprises:
a hollow main body adapted and configured to be coaxially mounted around the body of the pen injection system, the hollow main body comprising a central longitudinal bore having a proximal extremity and a distal extremity, and a central longitudinal axis;
a magnetic field production means, located on or within the hollow main body, at the proximal extremity of the central longitudinal bore;
an injection monitoring system comprising at least one or a plurality of magnetic sensors, the injection monitoring system being located at the proximal extremity of the bore of the hollow main body;
the hollow main body further comprising an inner sleeve located within the central longitudinal bore, and the inner sleeve is configured to frictionally engage with an outer surface of the dose setting wheel to co-rotate around the central longitudinal axis, without axial translation along said central longitudinal axis, with the dose setting wheel during during dose setting;
wherein the inner sleeve is connected to the injection monitoring system, and the connection between the inner sleeve and the injection monitoring system is adapted and configured to co-rotate both the inner sleeve and injection monitoring system about the central longitudinal axis during dose setting, and to translate the injection monitoring system along the central longitudinal axis, but not rotate said injection monitoring system around said central longitudinal axis, during injection and/or ejection of a drug from the pen injection system;
wherein the hollow main body further comprises a distal body portion which extends around an outer surface of the body of the injection pen system at a location distal to the dose setting wheel, said distal body portion comprising diameter altering means configured to dynamically alter an inner diameter of the central longitudinal bore of the distal body portion from a first value to a second value different to the first value.

2. Injection monitoring module according to claim 1, wherein the diameter altering means of the distal body portion are configured to dynamically alter the inner diameter of the central longitudinal bore of the distal body portion from a first value to a second value smaller than the first value.

3. Injection monitoring module according to claim 1, wherein the diameter altering means of the distal body portion are configured to dynamically alter the inner diameter of the central longitudinal bore of the distal body portion from a first value to a second value greater than the first value.

4. Injection monitoring module according to claim 1, wherein the diameter altering means of the distal body portion are configured to dynamically alter the inner diameter of the central longitudinal bore of the distal body portion from a first diameter in which, when mounted on the injection pen device, the distal body portion is not in frictional contact with an outer surface of the injection pen device, to a second diameter, smaller than the first diameter, in which the distal body portion is in frictional contact with the outer surface of the injection pen device, said frictional contact preventing axial movement of the distal body portion along the outer surface of the injection pen device.

5. Injection monitoring module according to claim 1, wherein the diameter altering means of the distal body portion comprises a first, rotatable outer component, and a second, non-rotatable inner component, wherein the diameter altering means is configured to dynamically alter the inner diameter of the central longitudinal bore of the distal body portion via a rotation of the first, rotatable outer component around and against the second, non-rotatable inner component.

6. Injection monitoring module according to claim 1, wherein the first, rotatable outer component is a rigid outer ring.

7. Injection monitoring module according to claim 1, wherein the second, non-rotatable inner component is a deformable inner ring.

8. Injection monitoring module according to claim 7, wherein the deformable inner ring comprises a radially inward-facing surface portion of elastomeric material.

9. Injection monitoring module according to claim 8, wherein the radially inward-facing surface portion of elastomeric material has a first thickness at a first point along a radius of curvature of the inward-facing surface portion, and a second thickness different to the first thickness at a second point along the radius of curvature of the inward facing surface portion.

10. Injection monitoring module according to claim 5, wherein the first, rotatable outer component comprises at least one, or a plurality of, radially inwardly facing portion(s), and said one, or plurality of radially inwardly facing portion(s) are configured to bear down on a radially outward facing surface of the second, non-rotatable inner component and deform said inner component when said first, rotatable outer component is rotated around and against said second, non-rotatable inner component.

11. Injection monitoring module according to claim 10, wherein the at least one, or a plurality of, radially inwardly facing portion(s) of the first, rotatable outer component, configured to bear down on the radially outward facing surface of the second, non-rotatable inner component, are spaced equally one from each other around an inner circumference of the first, rotatable outer component.

12. Injection monitoring module according to claim 10 wherein the at least one, or a plurality of, radially inwardly facing portion(s) of the first, rotatable outer component comprises three radially inwardly facing portions spaced equally, one from each other.

13. Injection monitoring module according to claim 10, wherein the radially outward facing surface of the second, non-rotatable inner component of the diameter altering means is configured to receive the at least one, or the plurality of, radially inwardly facing portion(s) of the first, rotatable outer component.

14. Injection monitoring module according to claim 10, wherein the radially outward facing surface of the second, non-rotatable inner component of the diameter altering means comprises an annular groove, configured to receive, and engage frictionally, with the at least one, or the plurality of, radially inwardly facing portion(s) of the first, rotatable outer component.

15. Injection monitoring module according to claim 14, wherein when rotated, the at least one, or the plurality of, radially inwardly facing portion(s) of the first, rotatable outer component engages with and bears against the annular groove of the radially outward facing surface of the second, non-rotatable inner component, and moves said at least one, or the plurality of, radially inwardly facing portion(s) of the first, rotatable outer component, about an axis of rotation of the first, rotatable outer component, from a first, radial position to a second, radial position which is different to the first radial position, whereby the inner diameter of the second, non-rotatable inner component is altered dynamically from a first inner diameter to a second inner diameter.

16. Injection monitoring module according to claim 5, wherein the first, rotatable outer component comprises an alignment marker, which is positioned on an outward facing surface of the first, rotatable outer component, and which is configured to move, when the first, rotatable outer component is rotated about an axis of rotation, from a first radial position of non-longitudinal alignment with an alignment marker located on an outward facing surface of the hollow main body, to a second radial position of longitudinal alignment with said alignment marker located on the outward facing surface of the hollow main body.

17. Injection monitoring module according to claim 5, wherein the distal portion comprises a positional feedback means configured to provide a positional feedback with regard to the rotation of the first, rotatable outer component about the central longitudinal axis, relative to the second, inner non-rotatable component.

18. Injection monitoring module according to claim 17, wherein the positional feedback means is configured to produce a zone of increased resistance to rotation, or a rotational stop, of the first, rotatable outer component, relative to the second, inner non-rotatable component.

19. Injection monitoring module according to claim 17, wherein the positional feedback means is configured to produce an audible signal, such as a click, to indicate that a rotational stop position of the first, rotatable outer component, relative to the second, inner non-rotatable component, has been reached.
